(19) United States
(12) Patent Application Publication
(10) Pub. No.: US 2025/0332381 A1
(43) Pub. Date: Oct. 30, 2025

(54) CATHETER AND CATHETER SYSTEM

(71) Applicant: TERUMO KABUSHIKI KAISHA, Tokyo (JP)

(72) Inventor: Mitsuki HOSHI, Irvine, CA (US)

(73) Assignee: TERUMO KABUSHIKI KAISHA, Tokyo (JP)

(21) Appl. No.: 19/262,786

(22) Filed: Jul. 8, 2025

Related U.S. Application Data
(63) Continuation of application No. PCT/JP2023/043758, filed on Dec. 7, 2023.

(30) Foreign Application Priority Data
Jan. 30, 2023 (JP) 2023-011752

(51) Int. Cl. A61M 25/00; A61M 25/01

(52) U.S. Cl. CPC A61M 25/0068; A61M 25/0108; A61M 2205/32

(57) ABSTRACT

An antegrade catheter of a catheter system includes a first marker having a hollow shape and disposed at a distal portion of a first catheter body. The first marker is formed in a tapered shape whose diameter decreases in a distal direction of the first catheter body. A retrograde catheter of the catheter system includes a second marker having a hollow shape and disposed at a distal portion of a second catheter body. The second marker is formed in a tapered shape whose diameter decreases in a distal direction of the second catheter body.

### CROSS-REFERENCES TO RELATED APPLICATIONS

This application is a continuation of International Application No. PCT/JP2023/043758 filed on Dec. 7, 2023, which claims priority to Japanese Patent Application No. 2023-011752 filed on Jan. 30, 2023, the entire content of both of which is incorporated herein by reference.

### TECHNOLOGICAL FIELD

The present invention generally relates to a catheter to be inserted into a tubular cavity of a living body, and a catheter system.

### BACKGROUND DISCUSSION

JP 2010-279546 A discloses a catheter system including a radiopaque marker at a distal portion. The catheter system includes a catheter body having flexibility, and a radiopaque marker embedded in a distal portion of the catheter body. The radiopaque marker is a cylindrical body formed of a metal material.

When there is a lesion (stenosis) in a tubular cavity of a patient, a distal end of the catheter is advanced along the tubular cavity under angiography. A position of the distal end of the catheter is checked with the radiopaque marker, and the distal end of the catheter is delivered to the lesion to treat the lesion.

### SUMMARY

When lower limb blood vessel treatment is performed on a lesion of a lower limb artery of a patient, for example, an antegrade catheter is inserted into a blood vessel in an antegrade approach first, and then another retrograde catheter is inserted into the blood vessel in a retrograde approach from a direction opposite to the antegrade catheter. By inserting a distal end of the another retrograde catheter into the distal end of the antegrade catheter in the vicinity of the lesion, the distal ends of the antegrade catheter and the retrograde catheter are disposed at a predetermined position in the vicinity of the lesion, and a balloon catheter is delivered to the lesion through the antegrade catheter.

The catheter disclosed in JP 2010-279546 A is formed in a reverse tapered shape in which the radiopaque marker expands in diameter toward the distal end of the catheter body, so that a diameter difference between the distal end of the catheter body and the distal end of the radiopaque marker is large. Therefore, when the catheter of JP 2010-279546 A is used as the antegrade catheter and the retrograde catheter, under angiography, it is difficult to insert the distal end of the retrograde approach while being aligned with the distal end of the catheter inserted in the antegrade approach while the distal end position of the radiopaque marker is being checked.

In addition, when the distal end of the catheter enters a complex lesion accompanied by calcification, the distal end of the catheter may be damaged due to contact between the hardened lesion and the distal end of the catheter.
(1) A first aspect of the present invention is a catheter that is inserted into a tubular cavity of a living body and is able to advance along the tubular cavity, the catheter including: a catheter body having a tubular shape and having a lumen; and a marker having a hollow shape, containing a radiopaque material, and disposed at a distal portion of the catheter body, in which an outer peripheral surface of the distal portion of the catheter body has a tapered outer surface portion whose diameter decreases in a distal direction, and the marker is formed in a tapered shape in the distal direction of the catheter body.

According to this catheter, when the catheter is advanced along the tubular cavity of the living body, a distal end position of the catheter can be accurately checked under angiography by using the marker. Since the distal portion of the catheter body and the marker are both formed in a tapered shape in the distal direction, a diameter difference between the distal portion of the catheter and the marker is small, and a distal end position of the catheter can be checked more accurately. A strength of the distal end of the catheter can be increased by the marker, and an outer diameter of the distal end of the catheter can be made thinner and sharper, so that a penetrating force of the catheter with respect to a stenosis is improved.
(2) In the catheter according to (1), the catheter may be an antegrade catheter that advances along the tubular cavity to a peripheral side of the living body, or a retrograde catheter that advances along the tubular cavity to a central side of the living body and has a distal end insertable into a distal end of the antegrade catheter.

With this configuration, by providing the marker at the distal end of the antegrade catheter or the distal end of the retrograde catheter, it is easy to align the distal end of the antegrade catheter and the distal end of the retrograde catheter during a technique in which the antegrade catheter and the retrograde catheter are moved towards one another and positioned in partial axially overlapping relation to each other. This technique is referred to as a rendezvous technique.
(3) In the catheter according to (1) or (2), a distance between a distalmost end of the marker and a distalmost end of the catheter body in an axial direction of the catheter body may be 0.5 mm or less.

With this configuration, since a distance between the distalmost end of the marker and the distalmost end of the catheter body in the axial direction is 0.5 mm or less, the distalmost end position of the catheter can be accurately checked under angiography.
(4) In the catheter according to any one of (1) to (3), the marker may have a plurality of grooves provided at least at a distal end of the marker, recessed radially inward from an outer peripheral surface of the marker, and provided in a circumferential direction of the marker about an axis of the marker, and, in the marker, a cross-sectional shape of the marker orthogonal to the axis of the marker may change from the distal end toward a proximal end in the marker.
(5) In the catheter according to (4), the cross-sectional shape of the marker may change when a number of the grooves in a proximal portion of the marker is smaller than a number of the grooves in a distal portion of the marker.
(6) In the catheter according to (4) or (5), the cross-sectional shape of the marker may change when a depth of each of the grooves with respect to the outer peripheral surface of the marker in a proximal portion of the marker is shallower than a depth of each of the grooves with respect to the outer peripheral surface of the marker in a distal portion of the marker.
(7) In the catheter according to any one of (1) to (6), a surface roughness of an outer peripheral surface of the marker may be smaller at a proximal portion of the marker than a surface roughness at a distal portion of the marker.

With this configuration, when the catheter is advanced along the tubular cavity of the living body, contact resistance between the proximal portion of the marker having the largest diameter and the tubular cavity can be reduced, so that the catheter can be smoothly advanced.
(8) A second aspect of the disclosure involves a catheter system including a catheter that is inserted into a tubular cavity of a living body and is able to advance along the tubular cavity, the catheter system including: an antegrade catheter that advances along the tubular cavity to a peripheral side of the living body; and a retrograde catheter that advances along the tubular cavity to a central side of the living body, in which the antegrade catheter includes: a first catheter body having a tubular shape and having a first lumen; and a first marker having a hollow shape, containing a radiopaque material, and disposed at a distal portion of the first catheter body, the distal portion of the first catheter body is formed in a tapered shape in a distal direction of the first catheter body, the first marker is formed in a tapered shape in a distal direction of the first catheter body, a distal end of the retrograde catheter is insertable into a distal end of the antegrade catheter, the retrograde catheter includes: a second catheter body having a tubular shape and having a second lumen; and a second marker having a hollow shape, containing a radiopaque material, and disposed at a distal portion of the second catheter body, the distal portion of the second catheter body is formed in a tapered shape in a distal direction of the second catheter body, and the second marker is formed in a tapered shape in a distal direction of the second catheter body.

According to this catheter system, when the antegrade catheter and the retrograde catheter are advanced along the tubular cavity of the living body and the distal end of the retrograde catheter is inserted into the distal end of the antegrade catheter, distal end positions of the first and second catheter bodies can be accurately checked under angiography by using the first and second markers. Since the first marker is formed in a tapered shape in the distal direction, a diameter difference between the distal portion of the first catheter body and the first marker is small. Since the second marker is formed in a tapered shape in the distal direction, a diameter difference between the distal portion of the second catheter body and the second marker is small. As a result, the distal end positions of the first and second catheter bodies can be checked more accurately, and the distal end of the retrograde catheter can be reliably inserted into the distal end of the antegrade catheter.

According to the disclosure here, since the catheter includes the marker having a hollow shape and disposed at the distal portion of the catheter body, the distal end position of the catheter can be accurately checked under angiography by using the marker when the catheter is advanced along the tubular cavity of the living body. Since the distal portion of the catheter body and the marker are both formed in a tapered shape in the distal direction, a diameter difference between the distal portion of the catheter and the marker is small, and a distal end position of the catheter can be checked more accurately. The marker can increase a strength of the distal end of the catheter.

In accordance with another aspect, a catheter that is insertable into a tubular cavity of a living body and that is advanceable along the tubular cavity includes an elongated catheter body, a hub, and a marker. The elongated catheter body extends in a distal direction from a proximalmost portion of the elongated catheter body to a distalmost portion of the elongated catheter body, the distalmost portion of the elongated catheter body terminates at a distalmost end of the elongated catheter body, and the proximalmost portion of the elongated catheter body terminates at a proximalmost end of the elongated catheter body. The hub is connected to the proximalmost portion of the elongated catheter body, and the elongated catheter body includes a lumen that extends throughout the elongated catheter body from the proximalmost end of the elongated catheter body to the distalmost end of the elongated catheter. The elongated catheter body has an outer peripheral surface extending from the proximalmost end of the elongated catheter body to the distalmost end of the elongated catheter body, and the distalmost portion of the elongated catheter body includes a tapered portion in which the outer peripheral surface of the elongated catheter body tapers in a narrowing manner in the distal direction. The elongated catheter body is made of a resin material, and the marker is disposed at the tapered portion of the distalmost portion of the catheter body. The marker is hollow throughout a longitudinal extent of the marker, and the marker contains a radiopaque material that is visually identifiable under radiation imaging in the living body. The marker has an outer peripheral surface that extends from a distalmost end of the marker to a proximalmost end of the marker, and the marker also has an inner peripheral surface that extends from the distalmost end of the marker to the proximalmost end of the marker. The marker is embedded in the resin material so that the entirety of the inner peripheral surface of the marker and the entirety of the outer peripheral surface of the marker are covered by and in direct contact with the resin material. In addition, the outer peripheral surface of the marker is tapered in the distal direction so that the outer diameter of the marker decreases in the distal direction.

### BRIEF DESCRIPTION OF THE DRAWINGS

FIG. 1 is an overall configuration diagram of a catheter system according to one embodiment disclosed as an example of the new catheter system disclosed here.

FIG. 2 is an enlarged cross-sectional view illustrating a distal portion of an antegrade catheter.

FIG. 3A is a cross-sectional view taken along line IIIA-IIIA in FIG. 2. FIG. 3B is a cross-sectional view taken along line IIIB-IIIB of FIG. 2.

FIG. 4 is an enlarged cross-sectional view illustrating a distal portion of the retrograde catheter.

FIG. 5A is a cross-sectional view taken along line VA-VA in FIG. 4. FIG. 5B is a cross-sectional view taken along line VB-VB of FIG. 4.

FIG. 6 is an explanatory view illustrating an initial state when treatment is performed using the catheter system.

FIG. 7 is an explanatory view illustrating a state in which the distal portion of the antegrade catheter is delivered to an upstream end portion of a lesion (CTO).

FIG. 8 is an explanatory view illustrating a state in which the distal portion of the retrograde catheter is inserted into the distal portion of the antegrade catheter in the lesion (CTO).

FIG. 9 is an enlarged cross-sectional view illustrating a distal portion of an antegrade catheter according to a modification.

### DETAILED DESCRIPTION

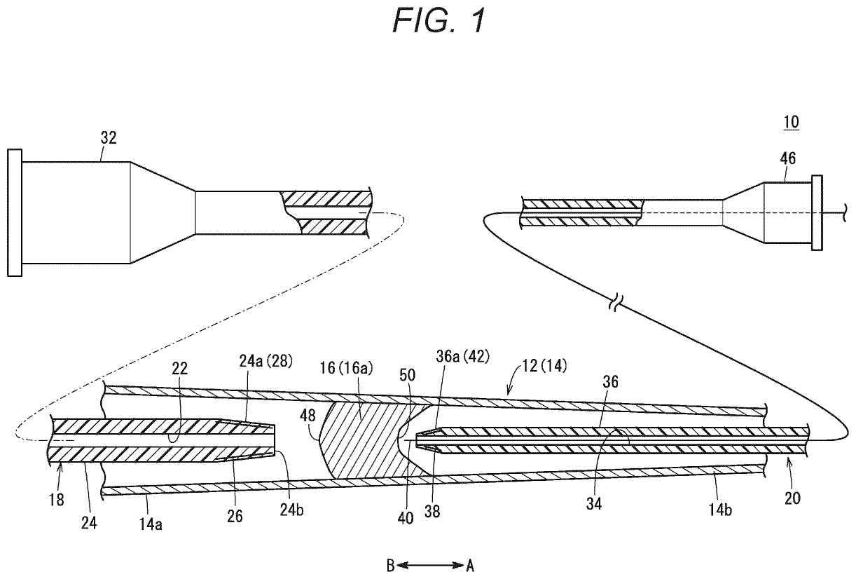

As illustrated in FIG. 1, a catheter system 10 according to the present embodiment is used, for example, for treatment of a lesion 16 (a stenosis, an occlusion, or the like) that has occurred or that is present in a blood vessel 14 of a living body 12. Specifically, the catheter system 10 is used for lower limb blood vessel treatment in which chronic total occlusion (CTO) 16a (lesion) that has occurred in the blood vessel 14 of a lower limb of the living body 12 is treated by an antegrade approach and a retrograde approach. The catheter system 10 may be for treating the lesion 16 in a tubular cavity other than the blood vessel 14, for example, in a living organ such as a bile duct, a trachea, an esophagus, a urethra, or other organs.

The catheter system 10 is inserted into the blood vessel 14 of the living body 12, and is able to advance along the blood vessel 14. The catheter system 10 has an antegrade catheter 18 for use in an antegrade approach in lower limb blood vessel treatment and another retrograde catheter 20 for use in a retrograde approach in lower limb blood vessel treatment.

The antegrade catheter 18 is a catheter that advances to a peripheral side (an ankle side, a direction of arrow A) of the living body 12 in a direction same as a blood flow along the blood vessel 14 of the living body 12 in lower limb blood vessel treatment.

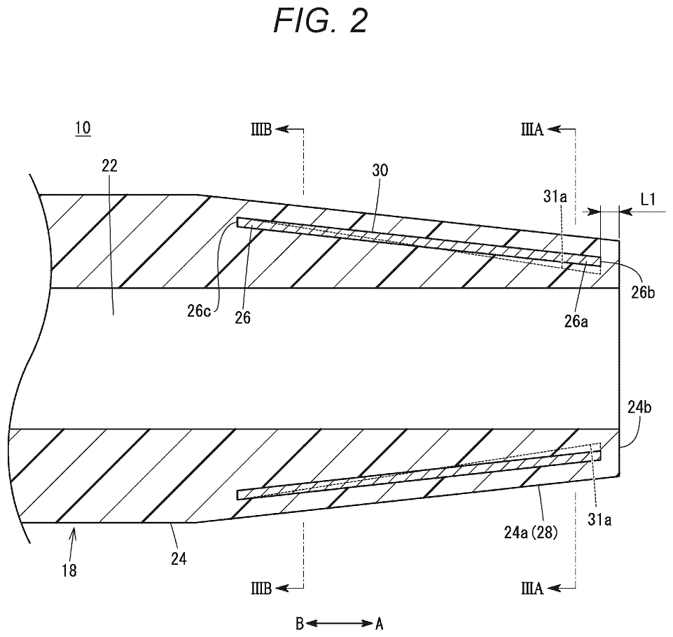

As illustrated in FIG. 2, the antegrade catheter 18 includes a first catheter body 24 having a tubular shape and having a first lumen 22, and a first marker 26 disposed at a distal portion 24a of the first catheter body 24.

The first catheter body 24 is formed of a flexible resin material. Specifically, as a constituent material from which the first catheter body 24 may be fabricated, a resin material having a certain degree of flexibility is used, such as a polyolefin such as polyethylene, polypropylene, and an ethylene-propylene copolymer, a polyester such as polyethylene terephthalate and polybutylene terephthalate, polystyrene, polyvinyl chloride, polyurethane, polyamide, or various elastomers such as a polyolefin elastomer, a polyester elastomer, a polyurethane elastomer, and a polyamide elastomer, which may be blended, layered, or disposed in multiple stages in an axial direction, and a reinforcing member obtained by braiding metal such as a stainless steel wire may be disposed. The first lumen 22 is disposed inside the first catheter body 24. The first lumen 22 extends along the first catheter body 24. Since the antegrade catheter 18 is used for treatment of the CTO 16a, the distal portion 24a of the first catheter body 24 is not made of a soft material such as a rubber material (elastomer material), and has hardness suitable for treatment of the CTO 16a.

The distal portion 24a of the first catheter body 24 is an end portion in an advancing direction when the antegrade catheter 18 is inserted into the blood vessel 14 and advanced. The distal portion 24a of the first catheter body 24 includes a first outer surface portion 28.

The first outer surface portion 28 is disposed on the outer peripheral surface of the first catheter body 24. The first outer surface portion 28 has a tapered shape whose diameter decreases in a distal direction (a direction of arrow A) of the first catheter body 24. The first outer surface portion 28 is disposed in a predetermined range from a distalmost end 24b toward a proximal end in the first catheter body 24. That is, the distal portion 24a of the first catheter body 24 gradually tapers in a narrowing manner toward the distalmost end 24b (the distal direction, the direction of arrow A).

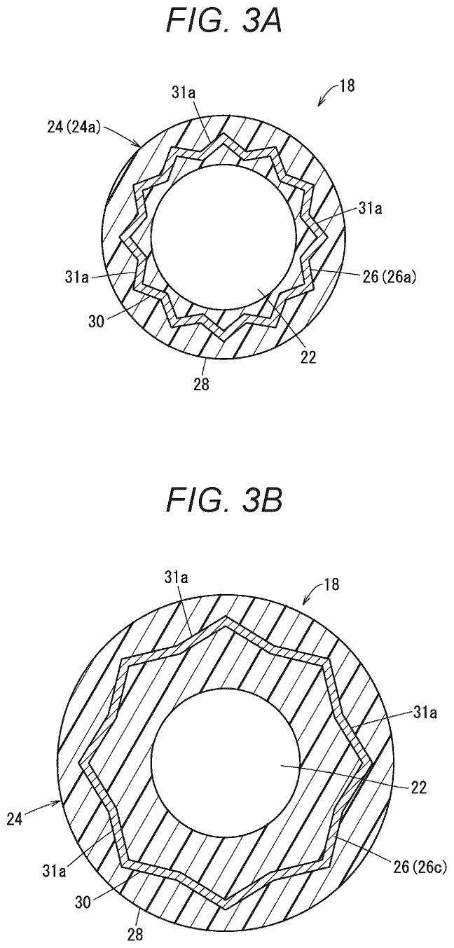

As illustrated in FIG. 2, the first marker 26 is formed in a hollow shape (see FIG. 3A) from a radiopaque metal material (for example, gold, platinum, iridium, tungsten, an alloy thereof, or the like). The first marker 26 enables a distal end position (distalmost end 24b) of the antegrade catheter 18 to be visually recognized under X-ray (radiation) imaging in the living body 12. The first marker 26 in the present embodiment is formed of a platinum iridium alloy.

The first marker 26 is embedded in the distal portion 24a of the first catheter body 24. The first marker 26 is formed in a tapered shape whose diameter decreases in the distal direction (the direction of arrow A) of the first catheter body 24. The first marker 26 is disposed along the first outer surface portion 28 of the first catheter body 24. That is, the first marker 26 is located at the portion of the first catheter body 24 at which the first outer surface portion 28 of the first catheter body 24 is located. The first outer surface portion 28 of the first catheter body 24 and the first marker 26 are substantially parallel to each other. A part of the first marker 26 may be exposed to the first outer surface portion 28.

A first outer peripheral surface 30 of the first marker 26 includes a plurality of marker-side grooves 31a. A cross-sectional shape of the first marker 26 orthogonal to an axial direction of the first marker 26 varies from the distal portion 26a toward a proximal portion 26c in the first marker 26.

As illustrated in FIGS. 3A and 3B, the plurality of marker-side grooves 31a are disposed apart from each other in a circumferential direction of the first marker 26 about an axis of the first marker 26. That is, the marker-side grooves 31a are circumferentially spaced apart from each other around the circumferential extent of the first marker 26. Each of the marker-side grooves 31a is recessed radially inward from the first outer peripheral surface 30 of the first marker 26. The number of the marker-side grooves 31a varies from the distal portion 26a toward the proximal portion 26c in the first marker 26. That is, the number of the marker-side grooves 31a in the distal portion 26a of the first marker 26 illustrated in FIG. 3A is the largest, and the number of the marker-side grooves 31a in the proximal portion 26c of the first marker 26 illustrated in FIG. 3B is the smallest. As a result, a cross-sectional shape orthogonal to the axial direction of the first marker 26 varies from the distal portion 26a toward the proximal portion 26c in the first marker 26.

As illustrated in FIG. 2, a radial depth of the marker-side groove 31a with respect to the first outer peripheral surface 30 of the first marker 26 varies from the distal portion 26a toward the proximal portion 26c in the first marker 26. A depth of the marker-side groove 31a in the distal portion 26a of the first marker 26 is the deepest, and a depth of the marker-side groove 31a in the proximal portion 26c of the first marker 26 is the shallowest. That is, as the depth of the marker-side groove 31a gradually decreases from the distal portion 26a toward the proximal portion 26c in the first marker 26, the cross-sectional shape orthogonal to the axial direction of the first marker 26 varies from the distal portion 26a toward the proximal portion 26c in the first marker 26.

The distal portion 26a of the first marker 26 has a distalmost end 26b disposed in a distalmost direction (the direction of arrow A). The distalmost end 26b of the first marker 26 is disposed on a proximal end side (a direction of arrow B) with respect to the distalmost end 24b of the first catheter body 24. In the axial direction (the directions of arrows A and B) of the first catheter body 24, an axial distance L1 between the distalmost end 26b of the first marker 26 and the distalmost end 24b of the first catheter body 24 is 0.5 mm or less. That is, the distalmost end 26b of the first marker 26 is disposed within 0.5 mm on the proximal end side (the direction of arrow B) from the distalmost end 24b of the first catheter body 24. The distalmost end 26b of the first marker 26 is not exposed to the outside from the distalmost end 24b of the first catheter body 24. An axial length of the first marker 26 is, for example, about 0.5 mm to 1.0 mm along an extending direction of the first catheter body 24.

As illustrated in FIG. 1, the proximal end of the first catheter body 24 includes a first hub 32. The first hub 32 has a hollow shape. A proximal end of the first hub 32 is open.

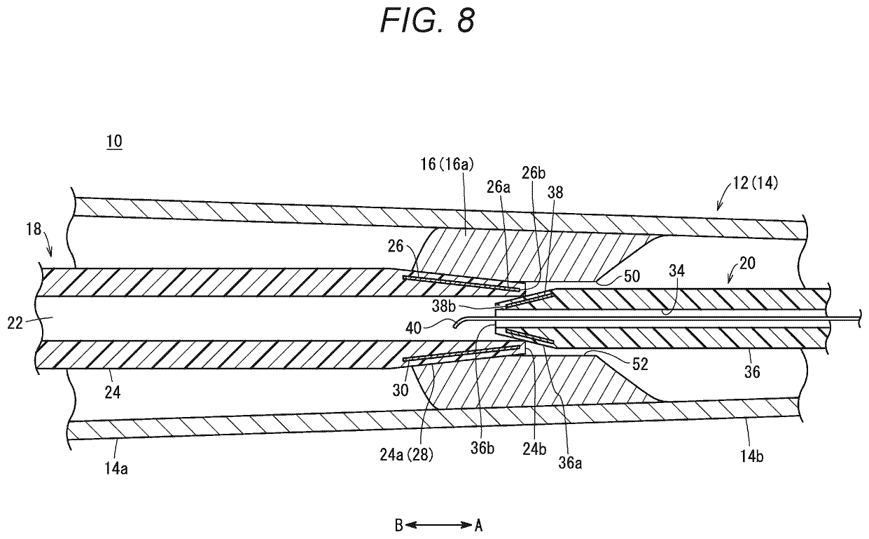

The retrograde catheter 20 advances to a central side (a heart side, the direction of arrow B) of the living body 12 in a direction opposite to a blood flow along the blood vessel 14 of the living body 12 in lower limb blood vessel treatment. When the retrograde catheter 20 advances toward the central side, the distal portion 36a of the retrograde catheter 20 can be inserted into the distal portion 24a of the antegrade catheter 18 (see FIG. 8) so that portions of the retrograde catheter 20 and the antegrade catheter 18 axially overlap one another.

The retrograde catheter 20 includes a second catheter body 36 having a tubular shape and having a second lumen 34, and a second marker 38 disposed at a distal portion 36a of the second catheter body 36.

The second catheter body 36 is formed of a flexible resin material. Specifically, as a constituent material from which the second catheter body 36 may be fabricated, a resin material having a certain degree of flexibility is used, such as a polyolefin such as polyethylene, polypropylene, and an ethylene-propylene copolymer, a polyester such as polyethylene terephthalate and polybutylene terephthalate, polystyrene, polyvinyl chloride, polyurethane, polyamide, or various elastomers such as a polyolefin elastomer, a polyester elastomer, a polyurethane elastomer, and a polyamide elastomer, which may be blended, layered, or disposed in multiple stages in an axial direction, and a reinforcing member obtained by braiding metal such as a stainless steel wire may be disposed. An outer diameter of the second catheter body 36 is smaller than an outer diameter of the first catheter body 24. The second lumen 34 is disposed inside the second catheter body 36. The second lumen 34 extends along the second catheter body 36. The second lumen 34 is a passage through which a guide wire 40 can be inserted. Since the retrograde catheter 20 is used for treatment of the CTO 16a, the distal portion 36a of the second catheter body 36 is not made of a soft material such as a rubber material (elastomer material), and has hardness suitable for treatment of the CTO 16a.

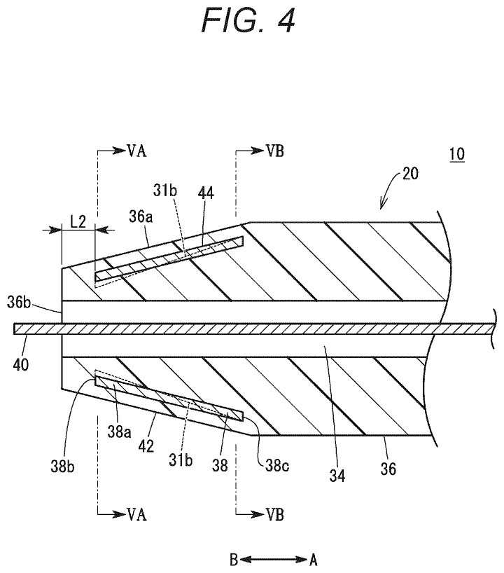

The distal portion 36a of the second catheter body 36 is an end portion in an advancing direction (the direction of arrow B) when the retrograde catheter 20 is inserted into the blood vessel 14 and advanced. As illustrated in FIG. 4, the distal portion 36a of the second catheter body 36 includes a second outer surface portion 42.

The second outer surface portion 42 is disposed on an outer peripheral surface of the second catheter body 36. The second outer surface portion 42 has a tapered shape whose diameter decreases in a distal direction (the direction of arrow B) of the second catheter body 36. The second outer surface portion 42 is disposed in a predetermined range from a distalmost end 36b toward a proximal end in the second catheter body 36. That is, the distal portion 36a of the second catheter body 36 gradually tapers in a narrowing manner toward the distalmost end 36b (the distal direction, the direction of arrow B).

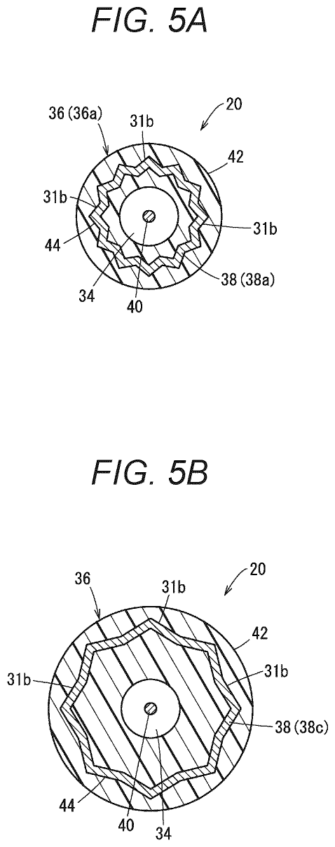

As illustrated in FIG. 4, the second marker 38 is formed in a hollow shape from a radiopaque metallic material (for example, gold, platinum, iridium, tungsten, or a mixture thereof) (see FIG. 5A). The second marker 38 enables a distal end position of the retrograde catheter 20 to be visually recognized (visually identifiable) under X-ray (radiation) imaging in the living body 12. The second marker 38 in the present embodiment is formed of a platinum iridium alloy.

The second marker 38 is embedded in the distal portion 36a of the second catheter body 36. The second marker 38 is formed in a tapered shape whose diameter decreases in the distal direction of the second catheter body 36. The second marker 38 is disposed along the second outer surface portion 42 of the second catheter body 36. That is, the second marker 38 is located at the portion of the second catheter body 36 at which is located the second outer surface portion 42 of the second catheter body 36. The second marker 38 and the second outer surface portion 42 of the second catheter body 36 are substantially parallel to each other. A part of the second marker 38 may be exposed to the second outer surface portion 42.

A second outer peripheral surface 44 of the second marker 38 includes a plurality of marker-side grooves 31b. A cross-sectional shape of the second marker 38 orthogonal to an axial direction of the second marker 38 varies from the distal portion 38a toward a proximal portion 38c in the second marker 38.

As illustrated in FIGS. 5A and 5B, the plurality of marker-side grooves 31b are disposed apart from each other in a circumferential direction of the second marker 38 about an axis of the second marker 38. That is, the marker-side grooves 31b are circumferentially spaced apart from each other around the circumferential extent of the second marker 38. Each of the marker-side grooves 31b is recessed radially inward from the second outer peripheral surface 44 of the second marker 38. The number of the marker-side grooves 31b varies from the distal portion 38a toward the proximal portion 38c in the second marker 38. That is, the number of the marker-side grooves 31b in the distal portion 38a of the second marker 38 illustrated in FIG. 5A is the largest, and the number of the marker-side grooves 31b in the proximal portion 38c of the second marker 38 illustrated in FIG. 5B is the smallest. As a result, a cross-sectional shape orthogonal to the axial direction of the second marker 38 varies from the distal portion 38a toward the proximal portion 38c in the second marker 38.

As illustrated in FIG. 4, a radial depth of the marker-side groove 31b with respect to the second outer peripheral surface 44 of the second marker 38 varies from the distal portion 38a toward the proximal portion 38c in the second marker 38. A depth of the marker-side groove 31b in the distal portion 38a of the second marker 38 is the deepest, and a depth of the marker-side groove 31b in the proximal portion 38c of the second marker 38 is the shallowest. That is, as the depth of the marker-side groove 31b gradually decreases from the distal portion 38a toward the proximal portion 38c in the second marker 38, the cross-sectional shape orthogonal to the axial direction of the second marker 38 varies from the distal portion 38a toward the proximal portion 38c in the second marker 38.

The distal portion 38a of the second marker 38 has a distalmost end 38b disposed in a distalmost direction (the direction of arrow B). The distalmost end 38b of the second marker 38 is disposed on a proximal end side (the direction of arrow A) with respect to the distalmost end 36b of the second catheter body 36. In the axial direction (the directions of arrows A and B) of the second catheter body 36, an axial distance L2 between the distalmost end 38b of the second marker 38 and the distalmost end 36b of the second catheter body 36 is 0.5 mm or less. That is, the distalmost end 38b of the second marker 38 is disposed within 0.5 mm on the proximal end side (the direction of arrow A) from the distalmost end 36b of the second catheter body 36. The distalmost end 38b of the second marker 38 is not exposed to the outside from the distalmost end 36b of the second catheter body 36. An axial length of the second marker 38 is, for example, about 0.5 mm to 1.0 mm along an extending direction of the second catheter body 36.

As illustrated in FIG. 1, the proximal end of the second catheter body 36 includes a second hub 46. The second hub 46 has a hollow shape. A proximal end of the second hub 46 is open. Through the second hub 46, the guide wire 40 can be inserted into the second lumen 34.

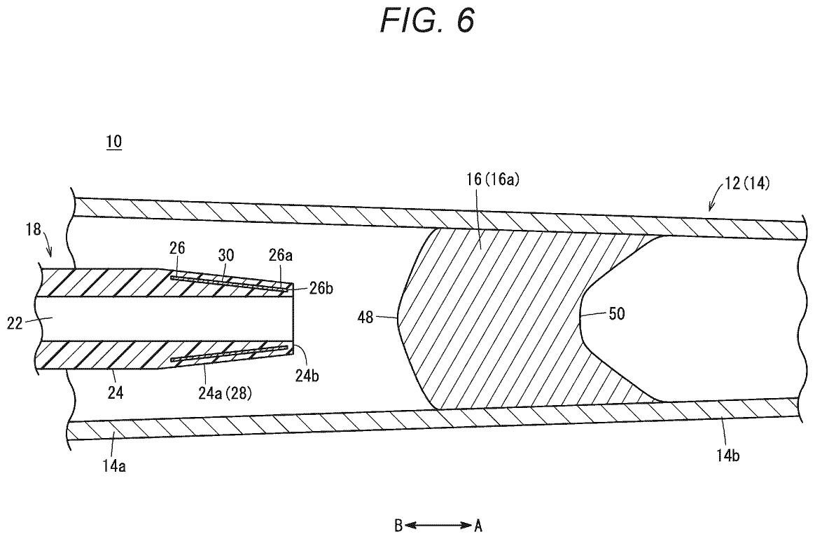

Next, a case of performing lower limb blood vessel treatment using the catheter system 10 will be described. FIG. 6 is a schematic cross-sectional view illustrating a peripheral portion of the CTO 16a (lesion 16) in a case where the CTO 16a occurs in the blood vessel 14 of the lower limb.

In FIG. 1, there is CTO 16a in the blood vessel 14 along an extending direction of the blood vessel 14, and a left side of the CTO 16a is a central side (heart side) and an upstream side of the blood flow. A right side of the CTO 16a is a peripheral side (ankle side) and a downstream side of the blood flow. Hereinafter, the blood vessel 14 on the upstream side (left side) with respect to the CTO 16a is referred to as an upstream blood vessel portion 14a, and the blood vessel 14 on the downstream side (right side) with respect to the CTO 16a is referred to as a downstream blood vessel portion 14b. The upstream blood vessel portion 14a is an artery having a relatively large tube diameter of the blood vessel 14. The downstream blood vessel portion 14b is a peripheral blood vessel having a smaller tube diameter of the blood vessel 14 than the upstream blood vessel portion 14a.

A diameter (outer diameter) of the antegrade catheter 18 is a diameter suitable for the tubular diameter (inner diameter) of the upstream blood vessel portion 14a. A diameter (outer diameter) of the retrograde catheter 20 is a diameter suitable for the tubular diameter (inner diameter) of the downstream blood vessel portion 14b. That is, although the diameter (outer diameter) of the antegrade catheter 18 is larger than the diameter (outer diameter) of the retrograde catheter 20, the diameter of the antegrade catheter 18 may be smaller than the diameter of the retrograde catheter 20 depending on a shape and hardness of the lesion 16. Alternatively, the antegrade catheter 18 may be used together with a single guide wire that has advanced retrogradely in a direction opposite to the blood flow, and the retrograde catheter 20 may be used together with a single guide wire that has advanced antegradely in a direction the same as the blood flow.

First, as illustrated in FIG. 6, the antegrade approach of the antegrade catheter 18 into the blood vessel 14 of the lower limb of the living body 12 is performed. A medical worker (not illustrated) percutaneously inserts the distal portion 24a of the antegrade catheter 18 into the upstream blood vessel portion 14a of the blood vessel 14. The distal portion 24a of the antegrade catheter 18 is advanced toward the CTO 16a and toward the peripheral side (the direction of arrow A) along the guide wire (not illustrated) in the blood vessel 14. At this time, the medical worker (not illustrated) can perform a procedure while visually recognizing a distal end position (the distalmost end 24b) of the antegrade catheter 18 by visually recognizing the first marker 26 through a display or the like under angiography. By using the first marker 26 having a hollow shape, the first marker 26 can be visually recognized when the distal portion 24a of the antegrade catheter 18 is viewed from any position in the circumferential direction (see FIG. 3A).

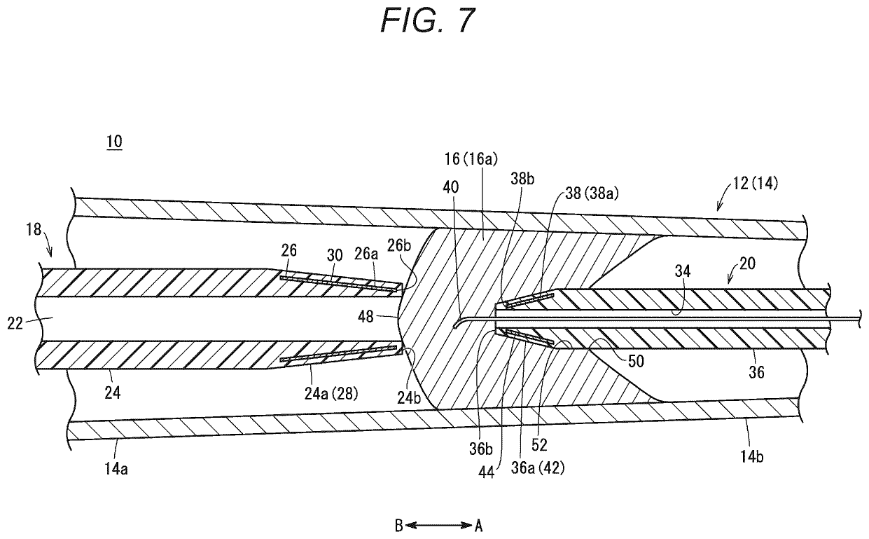

As illustrated in FIG. 7, along the upstream blood vessel portion 14a, the distal portion 24a (distalmost end 24b) of the antegrade catheter 18 is delivered to an upstream end portion of the CTO 16a. The upstream end portion of the CTO 16a has a protrusion 48 that is convex toward the upstream blood vessel portion 14a (the direction of arrow B). The distalmost end 24b of the antegrade catheter 18 contacts the protrusion 48. The medical worker (not illustrated) can check a position of the distal portion 24a of the antegrade catheter 18 with the first marker 26 on a display or the like.

After the distal portion 24a of the antegrade catheter 18 is delivered to the upstream end portion of the CTO 16a, the retrograde approach is taken in which the distal portion 36a of the retrograde catheter 20 is delivered to the CTO 16a along the downstream blood vessel portion 14b. However, the retrograde approach may be taken first (i.e., before the antegrade approach) in which the distal portion 36a of the retrograde catheter 20 is first delivered to the CTO 16a, and then the distal portion 24a of the antegrade catheter 18 may be delivered to the upstream end portion of the CTO 16a.

As illustrated in FIG. 1, the distal portion 36a of the retrograde catheter 20 is percutaneously inserted into the downstream blood vessel portion 14b of the blood vessel 14. At this time, the guide wire 40 is inserted through the second lumen 34 of the retrograde catheter 20. In a state where the distal end of the guide wire 40 protrudes in the distal direction (the direction of arrow B) from the distalmost end 36b of the retrograde catheter 20, the distal portion 36a of the retrograde catheter 20 is advanced toward the CTO 16a and toward the central side (the direction of arrow B) along the guide wire 40. The advancing direction (a first direction, toward the peripheral side) of the antegrade catheter 18 and the advancing direction (a second direction, toward the central side) of the retrograde catheter 20 are opposite directions. In other words, the distal portion 36a of the retrograde catheter 20 advances toward the distal portion 24a of the antegrade catheter 18.

Along the downstream blood vessel portion 14b, the distal portion 36a of the retrograde catheter 20 is delivered to a downstream end of the CTO 16a. A downstream end portion of the CTO 16a has a recess 50 recessed toward the upstream blood vessel portion 14a. The distalmost end 36b of the retrograde catheter 20 is inserted into the recess 50, and contacts a bottom portion of the recess 50. The bottom portion is on the most upstream blood vessel portion 14a side in the recess 50.

As illustrated in FIG. 7, by further advancing the distal portion 36a of the retrograde catheter 20, the distal portion 36a advances from the recess 50 to the inside of the CTO 16a. The distal portion 36a (distalmost end 36b) of the retrograde catheter 20 digs the CTO 16a to form a perforation 52. The perforation 52 is formed from the bottom portion of the recess 50 toward the protrusion 48. As the retrograde catheter 20 advances, the perforation 52 penetrates to the upstream end portion (protrusion 48) of the CTO 16a.

In a case where the upstream end portion of the CTO 16a has a convex shape (protrusion 48) toward the central side, it may be difficult to dig the CTO 16a toward the peripheral side with the antegrade catheter 18. Whereas, since there is a case where the downstream end portion of the CTO 16a has a concave shape (recess 50) toward the central side, it is easy to dig the CTO 16a toward the center side with the retrograde catheter 20, and it may be easy to advance in the CTO 16a or the vicinity of the center in the blood vessel 14.

The medical worker (not illustrated) checks a relative position between the first marker 26 of the antegrade catheter 18 and the second marker 38 of the retrograde catheter 20 under angiograhy, and aligns the first marker 26 and the second marker 38 so as to be arranged on a straight line along the extending direction (the directions of arrows A and B) of the blood vessel 14. By aligning the first marker 26 and the second marker 38, the distalmost end 24b of the antegrade catheter 18 and the distalmost end 36b of the retrograde catheter 20 are arranged on a straight line at the CTO 16a.

As illustrated in FIG. 8, by bringing the distal portion 24a of the antegrade catheter 18 and the distal portion 36a of the retrograde catheter 20 close to each other, the distal portion 24a (distalmost end 24b) of the antegrade catheter 18 advances inside the CTO 16a along the perforation 52. Inside the CTO 16a, the distal portion 36a of the retrograde catheter 20 is inserted into the first lumen 22 of the distal portion 24a of the antegrade catheter 18. At this time, since the perforation 52 is formed in the CTO 16a, the distal portion 24a of the antegrade catheter 18 is smoothly inserted into the CTO 16a along the perforation 52. The perforation 52 is expanded radially outward by the distal portion 24a of the antegrade catheter 18. The tapered first outer surface portion 28 of the antegrade catheter 18 enhances insertability of the antegrade catheter 18 into the perforation 52.

After the distal portion 24a of the antegrade catheter 18 is indwelled at a predetermined position inside the CTO 16a, the retrograde catheter 20 is removed. A balloon catheter (not illustrated) is inserted through the first lumen 22 of the antegrade catheter 18, and the balloon catheter is delivered to the CTO 16a. Treatment of the CTO 16a is performed with the balloon catheter. After an antegrade guide wire (not illustrated) is inserted into the first lumen 22 of the antegrade catheter 18, the antegrade catheter 18 may be removed, the antegrade guide wire may be indwelled, and the balloon catheter may be delivered to the CTO 16a along the antegrade guide wire (not illustrated). Alternatively, in a state where the antegrade guide wire (not illustrated) is indwelled, the antegrade catheter 18 may be removed, a guiding catheter having larger inner and outer diameters than those of the antegrade catheter 18 may be inserted, and then a treatment catheter such as a balloon catheter may be inserted into the guiding catheter and delivered to the CTO 16a to treat the lesion 16.

The insertion of the distal portion 36a of the retrograde catheter 20 into the distal portion 24a of the antegrade catheter 18 is not limited to the case of being performed inside the CTO 16a. The distal portion 36a of the retrograde catheter 20 may be inserted into the distal portion 24a of the antegrade catheter 18 in the upstream blood vessel portion 14a or the downstream blood vessel portion 14b in the vicinity of the CTO 16a. By inserting only the guide wire 40 into the distal portion 24a of the antegrade catheter 18, and coaxially disposing the guide wire 40, the first marker 26, and the second marker 38, the retrograde catheter 20 can be easily pushed. As a result, as long as the perforation 52 penetrates, it is not necessary to advance the distal portion 24a of the antegrade catheter 18 to a position where the retrograde catheter 20 is inserted.

The present embodiment has the following effects.

As illustrated in FIG. 1, the antegrade catheter 18 includes the first marker 26 having a hollow shape and disposed at the distal portion 24a of the first catheter body 24, and the outer peripheral surface of the distal portion 24a of the first catheter body 24 has the tapered first outer surface portion 28 whose diameter decreases in the distal direction. The first marker 26 is formed in a tapered shape in the distal direction of the first catheter body 24. As a result, when the antegrade catheter 18 is advanced along the blood vessel 14 (tubular cavity) of the living body 12, the distal end position of the antegrade catheter 18 can be accurately checked with the first marker 26 under angiography. Since the distal portion 24a of the first catheter body 24 and the first marker 26 are both formed in a tapered shape in the distal direction, a diameter difference between the distal portion of the antegrade catheter 18 and the first marker 26 is small, and a distal end position of the antegrade catheter 18 can be checked more accurately.

A strength of the distal end of the first catheter body 24 can be increased by the first marker 26 made of a radiopaque material (metal material). As a result, when the antegrade catheter 18 is advanced along the blood vessel 14 of the living body 12 and the distal end of the antegrade catheter 18 enters a complex lesion accompanied by calcification, the hardened lesion 16 (CTO 16a) can be suitably punctured by the distal end. When the distal end of the antegrade catheter 18 and the lesion 16 come into contact with each other, breakage of the distal portion 24a of the first catheter body 24 is prevented.

The retrograde catheter 20 includes the second marker 38 having a hollow shape and disposed at the distal portion 36a of the second catheter body 36, and the outer peripheral surface of the distal portion 36a of the second catheter body 36 has the tapered second outer surface portion 42 whose diameter decreases in the distal direction. The second marker 38 is formed in a tapered shape in the distal direction of the second catheter body 36. As a result, when the retrograde catheter 20 is advanced along the blood vessel 14 (tubular cavity) of the living body 12, the distal end position of the retrograde catheter 20 can be accurately checked by the second marker 38 under angiography. Since the distal portion 36a of the second catheter body 36 and the second marker 38 are both formed in a tapered shape in the distal direction, a diameter difference between the distal portion of the retrograde catheter 20 and the second marker 38 is small, and a distal end position of the retrograde catheter 20 can be checked more accurately.

A strength of the distal end of the second catheter body 36 can be increased by the second marker 38 made of a radiopaque material (metal material). As a result, when the retrograde catheter 20 is advanced along the blood vessel 14 of the living body 12 and the distal end of the retrograde catheter 20 enters a complex lesion accompanied by calcification, the hardened lesion 16 (CTO 16a) can be suitably punctured by the distal end. When the distal end of the retrograde catheter 20 and the lesion 16 come into contact with each other, breakage of the distal portion 36a of the second catheter body 36 is prevented.

As illustrated in FIG. 7, by providing the first marker 26 at the distal end of the antegrade catheter 18 and providing the second marker 38 at the distal end of the retrograde catheter 20, it is easy to align the distal end of the antegrade catheter 18 and the distal end of the retrograde catheter 20.

As illustrated in FIG. 2, the axial distance L1 between the distalmost end 26b of the first marker 26 disposed at the distal portion 24a of the antegrade catheter 18 and the distalmost end 24b of the first catheter body 24 is 0.5 mm or less. Therefore, when the antegrade catheter 18 is advanced toward the peripheral side (the direction of arrow A) along an inside of the blood vessel 14, the distal end position (distalmost end 24b) of the antegrade catheter 18 can be accurately checked under angiography. As illustrated in FIG. 4, the axial distance L2 between the distalmost end 38b of the second marker 38 disposed at the distal portion 36a of the retrograde catheter 20 and the distalmost end 36b of the second catheter body 36 is 0.5 mm or less. Therefore, when the retrograde catheter 20 is advanced toward the central side (the direction of arrow B) along an inside of the blood vessel 14, the distal end position (distalmost end 36b) of the retrograde catheter 20 can be accurately checked under angiography.

The first marker 26 has a plurality of marker-side grooves 31a provided at least at the distal end of the first marker 26, recessed radially inward from the first outer peripheral surface 30 of the first marker 26, and provided in the circumferential direction of the first marker 26 about the axis of the first marker 26. In the first marker 26, a cross-sectional shape of the first marker 26 orthogonal to the axis of the first marker 26 varies from the distal portion 26a toward the proximal portion 26c in the first marker 26. As a result, a recessed portion and a protruding portion are formed on the first outer peripheral surface 30 of the first marker 26 by the marker-side groove 31a, and the recessed portions or the protruding portions overlap each other more in the circumferential direction of the first marker 26 at the distal portion 26a than the proximal portion 26c of the first marker 26. As a result, the distal portion 26a has higher visibility under angiography than the proximal portion 26c of the first marker 26, and an orientation of the antegrade catheter 18 having the first marker 26 can be easily determined. Therefore, when performing an operation between the antegrade catheter 18 and the retrograde catheter 20 in which the antegrade catheter 18 and the retrograde catheter 20 are moved towards each other (i.e., a rendezvous technique), identification of the antegrade catheter 18 and the retrograde catheter 20 can be performed more easily.

The second marker 38 has a plurality of marker-side grooves 31b provided at least at the distal end of the second marker 38, recessed radially inward from the second outer peripheral surface 44 of the second marker 38, and provided in the circumferential direction of the second marker 38 about the axis of the second marker 38. In the second marker 38, a cross-sectional shape of the second marker 38 orthogonal to the axis of the second marker 38 varies from the distal portion 38a toward the proximal portion 38c in the second marker 38. As a result, a recessed portion and a protruding portion are formed on the second outer peripheral surface 44 of the second marker 38 by the marker-side groove 31b, and the recessed portions or the protruding portions overlap each other more in the circumferential direction of the second marker 38 at the distal portion 38a than the proximal portion 38c of the second marker 38. As a result, the distal portion 38a has higher visibility under angiography than the proximal portion 38c of the second marker 38, and an orientation of the retrograde catheter 20 having the second marker 38 can be easily determined. Therefore, when performing an operation between the antegrade catheter 18 and the retrograde catheter 20 in which the antegrade catheter 18 and the retrograde catheter 20 are moved towards each other (i.e., a rendezvous technique), identification of the antegrade catheter 18 and the retrograde catheter 20 can be performed more easily.

A depth of the marker-side groove 31a with respect to the first outer peripheral surface 30 of the first marker 26 in the proximal portion 26c of the first marker 26 is shallower than a depth of the marker-side groove 31a with respect to the first outer peripheral surface 30 of the first marker 26 in the distal portion 26a of the first marker 26. A cross-sectional shape of the first marker 26 varies from the distal end toward the proximal end in the first marker 26. A depth of the marker-side groove 31b with respect to the second outer peripheral surface 44 of the second marker 38 in the proximal portion 38c of the second marker 38 is shallower than a depth of the marker-side groove 31b with respect to the second outer peripheral surface 44 of the second marker 38 in the distal portion 38a of the second marker 38. A cross-sectional shape of the second marker 38 varies from the distal portion 38a toward the proximal portion 38c in the second marker 38.

As a result, a depth of the marker-side groove 31a is deeper in the distal portion 26a than that in the proximal portion 26c of the first marker 26. Therefore, the distal portion 26a of the first marker 26 has higher visibility under angiography than the proximal portion 26c of the first marker 26, and an orientation of the antegrade catheter 18 having the first marker 26 can be easily determined. Since a depth of the marker-side groove 31b is deeper in the distal portion 38a than that in the proximal portion 38c of the second marker 38, the distal portion 38a of the second marker 38 has higher visibility under angiography than the proximal portion 38c of the second marker 38, and an orientation of the retrograde catheter 20 having the second marker 38 can be easily determined. Therefore, when performing rendezvous between the antegrade catheter 18 and the retrograde catheter 20, identification of the antegrade catheter 18 and the retrograde catheter 20 can be performed more easily.

As illustrated in FIGS. 3A and 3B, since the number of the marker-side grooves 31a in the proximal portion 26c of the first marker 26 is smaller than the number of the marker-side grooves 31a in the distal portion 26a of the first marker 26, a cross-sectional shape of the first marker 26 varies from the distal portion 26a toward the proximal portion 26c in the first marker 26. Since the number of the marker-side grooves 31b in the proximal portion 38c of the second marker 38 is smaller than the number of the marker-side grooves 31b in the distal portion 38a of the second marker 38, a cross-sectional shape of the second marker 38 varies from the distal portion 38a toward the proximal portion 38c in the second marker 38. As a result, since the orientations (directions) of the distal portion 26a and the proximal portion 26c of the first marker 26 can be identified by using the number of the marker-side grooves 31a, the orientation of the antegrade catheter 18 can be easily identified. Since the orientations (directions) of the distal portion 38a and the proximal portion 38c of the second marker 38 can be identified by using the number of the marker-side grooves 31b, the orientation of the retrograde catheter 20 can be easily identified. Therefore, when performing rendezvous between the antegrade catheter 18 and the retrograde catheter 20, identification of the antegrade catheter 18 and the retrograde catheter 20 can be performed more easily.

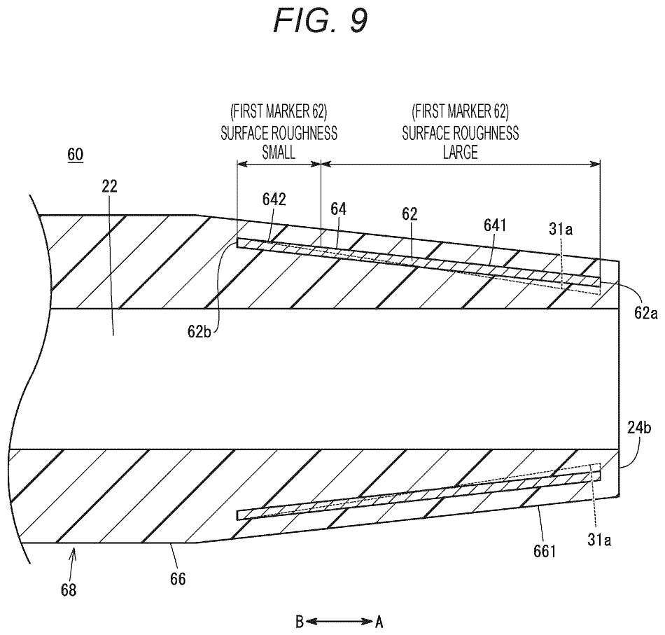

A first marker 62 of a catheter system 60 according to a modification illustrated in FIG. 9 includes first and second surfaces 641 and 642 having different surface roughnesses on a first outer peripheral surface 64. Hereinafter, as illustrated in FIG. 9, an antegrade catheter 68 having the first marker 62 and a first catheter body 66 will be described, but the same configuration can be applied to a retrograde catheter having a second marker.

The first marker 62 is embedded in the first outer surface portion 661. The first marker 62 is provided in a first outer surface portion 661 of the first catheter body 66. That is, the first marker 62 is located at the portion of the first catheter body 66 at which the first outer surface portion 661 of the first catheter body 66 is located. The first marker 62 is embedded and so the first outer peripheral surface 64 of the first marker 62 is not exposed to an outside of the first catheter body 66. The first and second surfaces 641 and 642 are provided circumferentially outside the first outer peripheral surface 64 of the first marker 62.

The first surface 641 is provided, for example, on the first outer peripheral surface 64 in a range from a distal portion 62a to the vicinity of a proximal end in the first marker 62. A surface roughness of the first marker 62 gradually decreases from the distal portion 62a toward a proximal portion 62b in the first marker 62. A surface roughness of the first surface 641 may be substantially uniform in an axial direction of the first marker 62.

The second surface 642 is disposed in a proximal direction of the first surface 641, and constitutes the first outer peripheral surface 64 together with the first surface 641. The second surface 642 extends from the proximal portion 62b toward the distal end in the first marker 62. The second surface 642 is disposed only in the vicinity of the proximal portion 62b of the first marker 62. A surface roughness of the second surface 642 may be smaller than a surface roughness of the first surface 641, or may be the same as the surface roughness of the first surface 641. In other words, the surface of the second surface 642 may be smoother than the surface of the first surface 641, and the surface of the second surface 642 and the surface of the first surface 641 may be substantially uniform.

The modification has the following effects.

In the catheter system 60 illustrated in FIG. 9, when the antegrade catheter 68 is advanced along the blood vessel 14 of the living body 12, in a case where the surface roughness of the first surface 641 increases toward the distal portion 62a of the first marker 62 from the second surface 642 disposed at the proximal portion 62b of the first marker 62 having a tapered shape and the largest diameter, accordingly, it is possible to suppress contact resistance between the first outer surface portion 661 and the blood vessel 14 at a proximal portion of the first catheter body 66. Therefore, the antegrade catheter 68 (first catheter body 66) can be smoothly advanced along the blood vessel 14.

The detailed description above describes embodiments of a catheter and catheter system representing examples of the new catheter and catheter system disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A catheter that is insertable into a tubular cavity of a living body and is advanceable along the tubular cavity, the catheter comprising:
an elongated catheter body that extends in a distal direction from a proximalmost portion of the elongated catheter body to a distalmost portion of the elongated catheter body, the distalmost portion of the elongated catheter body terminating at a distalmost end of the elongated catheter body, the proximalmost portion of the elongated catheter body terminating at a proximalmost end of the elongated catheter body;
a hub connected to the proximalmost portion of the elongated catheter body;
the elongated catheter body including a lumen that extends throughout the elongated catheter body from the proximalmost end of the elongated catheter body to the distalmost end of the elongated catheter;
the elongated catheter body having an outer peripheral surface extending from the proximalmost end of the elongated catheter body to the distalmost end of the elongated catheter body, the distalmost portion of the elongated catheter body including a tapered portion in which the outer peripheral surface of the elongated catheter body tapers in a narrowing manner in the distal direction;
the elongated catheter body being made of a resin material;
a marker disposed at the tapered portion of the distalmost portion of the catheter body, the marker being hollow throughout a longitudinal extent of the marker, the marker containing a radiopaque material that is visually identifiable under radiation imaging in the living body;
the marker having an outer peripheral surface that extends from a distalmost end of the marker to a proximalmost end of the marker, the marker also having an inner peripheral surface that extends from the distalmost end of the marker to the proximalmost end of the marker;
the marker being embedded in the resin material so that an entirety of the inner peripheral surface of the marker and an entirety of the outer peripheral surface of the marker are covered by and in direct contact with the resin material; and
the outer peripheral surface of the marker being tapered in the distal direction so that an outer diameter of the marker decreases in the distal direction.

2. The catheter according to claim 1, wherein the distalmost end of the marker is spaced from the distalmost end of the catheter body by an axial distance so that the distalmost end of the marker is on a proximal side of the distalmost end of the catheter body, the axial distance being 0.5 mm or less.

3. The catheter according to claim 1, wherein the marker, in a cross-section orthogonal to a central axis of the marker, includes a plurality of grooves that are recessed radially inward and that are circumferentially spaced apart from one another.

4. The catheter according to claim 1, wherein a cross-sectional shape of the marker, orthogonal to a central axis of the marker, varies along the longitudinal extent of the marker.

5. A catheter that is insertable into a tubular cavity of a living body and is advanceable along the tubular cavity, the catheter comprising:
a catheter body that has a tubular shape and includes a lumen extending along the catheter body, the catheter body having an outer peripheral surface and a distal portion;
a marker disposed at the distal portion of the catheter body, the marker having a hollow shape and containing a radiopaque material;
the outer peripheral surface of the distal portion of the catheter body being tapered so that an outer diameter of the outer peripheral surface of the distal portion of the catheter body decreases in a distal direction; and
the marker having a tapered shape in the distal direction of the catheter body.

6. The catheter according to claim 5, wherein
the catheter is an antegrade catheter that advances along the tubular cavity to a peripheral side of the living body, or a retrograde catheter that advances along the tubular cavity to a central side of the living body and that has a distal end insertable into a distal end of the antegrade catheter.

7. The catheter according to claim 5, wherein
a distance between a distalmost end of the marker and a distalmost end of the catheter body in an axial direction of the catheter body is 0.5 mm or less.

8. The catheter according to claim 5, wherein
the marker has a plurality of grooves provided at least at a distal end of the marker, recessed radially inward from an outer peripheral surface of the marker, and provided in a circumferential direction of the marker about an axis of the marker, and
a cross-sectional shape of the marker orthogonal to the axis of the marker varies from the distal end toward a proximal end in the marker.

9. The catheter according to claim 8, wherein
the cross-sectional shape of the marker varies by virtue of a number of the grooves in a proximal portion of the marker being smaller than a number of the grooves in a distal portion of the marker.

10. The catheter according to claim 9, wherein
the cross-sectional shape of the marker varies by virtue of a depth of each of the grooves with respect to the outer peripheral surface of the marker in the proximal portion of the marker is shallower than a depth of each of the grooves with respect to the outer peripheral surface of the marker in the distal portion of the marker.

11. The catheter according to claim 5, wherein a surface roughness of an outer peripheral surface of the marker is smaller at a proximal portion of the marker than a surface roughness at a distal portion of the marker.

12. The catheter according to claim 5, wherein the first catheter body is made of resin material, the first marker being embedded in the first catheter body so that inner and outer peripheral surfaces of the first marker are covered by the resin material.

13. A catheter system that is insertable into a tubular cavity of a living body and is advanceable along the tubular cavity, the catheter system comprising:
an antegrade catheter that is advanceable along the tubular cavity to a peripheral side of the living body;
a retrograde catheter that is advanceable along the tubular cavity to a central side of the living body;
the antegrade catheter comprising:
a first catheter body that has a tubular shape and that includes a first lumen extending along the first catheter body, the first catheter body having an outer peripheral surface and a distal portion;
a first marker disposed at the distal portion of the first catheter body, the first marker having a hollow shape and containing a radiopaque material;
the distal portion of the first catheter body being tapered in a distal direction of the first catheter body so that an outer diameter of the distal portion of the first catheter body decreases in the distal direction;
the first marker having a tapered shape in a distal direction of the first catheter body so that an outer diameter of the first marker decreases in the distal direction of the first catheter body; and
a distal end of the retrograde catheter being configured to be insertable into a distal end of the antegrade catheter,
the retrograde catheter comprising:
a second catheter body that has a tubular shape and includes a second lumen extending along the second catheter body, the second catheter body having an outer peripheral surface and a distal portion;
a second marker disposed at the distal portion of the second catheter body, the second marker having a hollow shape and containing a radiopaque material;
the distal portion of the second catheter body being tapered shape in a distal direction of the second catheter body so that an outer diameter of the distal portion of the second catheter body decreases in the distal direction, and
the second marker having a tapered shape in a distal direction of the second catheter body so that an outer diameter of the second marker decreases in the distal direction of the second catheter body.

14. The catheter system according to claim 13, wherein a distance between a distalmost end of the marker and a distalmost end of the catheter body in an axial direction of the catheter body is 0.5 mm or less.

15. The catheter system according to claim 13, wherein the first marker includes a plurality of grooves at least at a distal end of the first marker, the grooves of the first marker being recessed radially inward and being circumferentially spaced apart from one another.

16. The catheter system according to claim 15, wherein the second marker includes a plurality of grooves at least at a distal end of the second marker, the grooves of the second marker being recessed radially inward and being circumferentially spaced apart from one another.

17. The catheter system according to claim 13, wherein the second marker includes a plurality of grooves at least at a distal end of the second marker, the grooves of the second marker being recessed radially inward and being circumferentially spaced apart from one another.

18. The catheter system according to claim 13, wherein the first marker has a cross-sectional shape orthogonal to a central axis of the first marker that varies from a distal end of the first marker toward a proximal end of the first marker.

19. The catheter system according to claim 18, wherein the second marker has a cross-sectional shape orthogonal to a central axis of the second marker that varies from a distal end of the second marker toward a proximal end of the second marker.

20. The catheter system according to claim 13, wherein the second marker has a cross-sectional shape orthogonal to a central axis of the second marker that varies from a distal end of the second marker toward a proximal end of the second marker.
